(19) United States
(12) Patent Application Publication
(10) Pub. No.: US 2020/0211717 A1
(43) Pub. Date: Jul. 2, 2020

(54) Systems and Methods for Visualizing Patient Population Disease Symptom Comparison

(71) Applicant: CURELATOR, INC., Palmer, MA (US)

(72) Inventors: Marc ALBERT, Palmer, MA (US); Gabriel BOUCHER, Palmer, MA (US); Alec MIAN, Palmer, MA (US)

(73) Assignee: Curelator, Inc., Palmer, MA (US)

(21) Appl. No.: 16/619,863

(22) PCT Filed: Jun. 11, 2018
(86) PCT No.: PCT/US2018/036956

§ 371 Date: Dec. 5, 2019

Related U.S. Application Data
(60) Provisional application No. 62/517,552, filed on Jun. 9, 2017.

(51) Int. Cl. G16H 50/80; G16H 50/70; G16H 70/60; A61B 5/00

(52) U.S. Cl. CPC G16H 50/80; G16H 50/70; A61B 5/7275; A61B 5/7264; G16H 70/60

(57) ABSTRACT

Embodiments disclosed herein include receiving disease symptom and disease factor inputs from a patient population comprising a plurality of patients, determining whether individual disease factors tend to cause individual patients in the patient population to experience individual disease symptoms or prevent individual patients in the patient population from experiencing individual disease symptoms, and causing a graphical user interlace to display a patient population trigger visualization for the disease symptoms, wherein the trigger visualization comprises a plurality of rows and one or more columns, wherein a first column corresponds to a first disease symptom for a first patient, and wherein a first row in the first column comprises an indication of an extent to which a first risk factor is a disease trigger or disease protector for the first disease symptom for the first patient.

### CROSS-REFERENCE TO RELATED APPLICATIONS

The application claims priority U.S. provisional application 62/517,552 filed on Jun. 9, 2018, titled “Systems and Methods for Visualizing Patient Population Disease Symptom Comparison,” and currently pending. This application incorporates the entire contents of U.S. provisional application 62/517,552 by reference. This application also incorporates the entire contents of the following applications by reference: (i) U.S. application Ser. No. 15/502,087, filed on Feb. 6, 2017; (ii) PCT App. PCT/US15/43945, filed on Aug. 6, 2015; (iii) U.S. provisional application 62/034,408 filed on Aug. 7, 2014; (iii) U.S. provisional application 62/120,534 filed on Feb. 25, 2015; (iv) U.S. provisional application 62/139,291 filed on Mar. 27, 2015; (v) U.S. provisional application 62/148,130 filed on Apr. 15, 2015; (vi) U.S. provisional application 62/172,594 filed on Jun. 8, 2015; and (vii) PCT App. PCT/US14/013894, filed on Jan. 30, 2014.

### SUMMARY

Medical researchers and/or patients may benefit from embodiments of the computer-based methods and systems described herein, which are configured to: (i) determine statistical associations and/or correlations between risk factors and disease symptoms for one or more patients, (ii) identify whether and the extent to which one or more risk factors tend to trigger or protect against one or more disease symptoms for one or more patients; and (iii) display via a graphical user interface, for one or more patients, one or more visualizations indicating, on a patient-by-patient basis, whether and the extent to which one or more risk factors tend to trigger or protect against one or more disease symptoms for one or more patients.

Some embodiments additionally or alternatively: (i) determine statistical associations and/or correlations between risk factors and the onset and/or severity of individual patient's disease symptoms, (ii) identify whether and the extent to which risk factors affect the onset and severity of a particular disease symptom for a particular patient and/or group of patients; and (iii) display via a graphical user interface, for one or more patients, one or more visualizations indicating, on a patient-by-patient basis, whether and the extent to which one or more risk factors affect the onset and severity of one or more particular disease symptoms.

As used herein, a disease symptom is a physical manifestation of a particular disease. A disease symptom can be characterized by multiple characterization metrics, including but not limited to one or more of: (i) a time (or range of times) when the patient experiences the disease symptom, which can be quantified and/or represented as an occurrence or frequency of occurrence; (ii) a severity of the disease symptom; (iii) aspects or characteristics describing the disease symptom; and/or (iv) whether the disease symptom is accompanied by other related disease symptoms (and perhaps risk factors and/or disease triggers/protectors as well).

In examples where the disease symptom is a migraine headache, the characterization metrics for the migraine headache may include any one or more of: (i) when the migraine headache occurred; (ii) how long the migraine headache lasted; (iii) the intensity and/or severity of the migraine headache; (iv) whether the migraine headache was accompanied by other related symptoms such as nausea or dizziness, and if so, the time, duration, intensity/severity of the accompanying symptoms. Disease symptoms for other chronic diseases may include different characterization metrics.

As used herein, a risk factor is any event, exposure, action, or conduct related to and/or performed by a patient that has the potential to influence, affect, or cause the patient to experience a disease symptom, prevent the patient from experiencing a disease symptom, and/or reduce or increase the severity of the disease symptom experienced by the patient. Disease factors may include both: (i) voluntary or modifiable conduct and/or experiences by the patient over which the patient has at least some control, such as consumption of a particular food product, ingestion of a particular therapeutic agent, application of a particular therapeutic agent, ingestion of a particular dietary supplement or drug, performance of a particular physical activity, and/or exposure to a particular chemical agent; and (ii) involuntary or un-modifiable conduct and/or experiences, such as exposure to environmental factors (e.g., smog, sunlight, rain, snow, high or low humidity, or high or low temperatures), ingestion or other exposure to mandatory therapeutic agents or drugs (e.g., drugs to maintain other diseases), and effects of other diseases or physical conditions over which the patient has little or perhaps effectively no control over.

Like a disease symptom, a risk factor can also be characterized by multiple characterization metrics, and different risk factors may have different characterization metrics. For example, for a food or drug consumption based risk factor, the characterization metrics may include, for example: (i) when the patient consumed the food or drug; and/or (ii) how much of the food or drug the patient consumed. Characterization metrics for an exposure based risk factor may include, for example: (i) when the patient was exposed; (ii) the intensity (e.g., bright sunlight) of the exposure; and/or (iii) the duration of the exposure.

In some embodiments, risk factors may also include premonitory symptoms or warning signs that may not actually cause the patient to experience a disease symptom, but may just be closely associated with onset of a disease symptom for a particular patient. To use the migraine example again, a premonitory symptom might be a craving for sweet foods perhaps caused by a chemical change in the patient's body before the patient experiences the migraine. The sweet craving does not cause the migraine, but instead is likely caused by some chemical change that also causes the patient to experience the migraine. Likewise, risk factors may also include postdrome symptoms between when the disease symptom ends (e.g., when the most intense and painful phase of migraine headache is over) and when the patient feels “back to normal” again.

In some instances, a particular physical manifestation felt by the patient may be a disease symptom or a risk factor depending on the context. To use the migraine example again, abnormal body temperature, abnormal heart rate, and abnormal blood sugar levels may be risk factors because they tend to cause a disease symptom such as migraine headache. But in other contexts, abnormal body temperature, abnormal heart rate, and abnormal blood sugar levels may be disease symptoms that are caused by other risk factors.

As used herein, a disease trigger is a risk factor that has been determined, for example through statistical analyses or other analytical method, to have a sufficiently strong association with causing the patient to experience the particular disease symptom, or at least increasing the risk or likelihood that the patient will experience the particular disease symptom. In some contexts, a disease trigger may one or both (i) increase the severity of a disease symptom, when experienced, and/or (ii) increase the likelihood of disease symptom onset in the first instance.

As used herein, a protector is a risk factor that has been determined, for example through statistical analyses or other analytical method, to have a sufficiently strong association with preventing the patient from experiencing the particular disease symptom, or at least reducing the risk or likelihood that the patient will experience the particular disease symptom. In some contexts, a protector may one or both (i) reduce the severity of a disease symptom, when experienced, and/or (ii) reduce the likelihood of disease symptom onset in the first instance.

In some embodiments, a disease trigger/protector for a patient is a risk factor having a determined univariate association with a disease symptom for the patient, where the determined univariate association has a Cox Hazard Ratio greater than 1 and a p-value less than or equal to 0.05.

In some embodiments, one or more server systems analyze disease symptom and risk factor data received from a patient population to determine which risk factors rise to the level of disease triggers/protectors for a particular patient. In operation, a patient population may include many (hundreds, thousands, or perhaps millions) of patients who all share one or more similarities (e.g., the same age or age range, same gender, same ethnicity, same national origin, suffer from the same disease, have the same allergies, have the same genetic markers, and/or perhaps other similarities). Some patients may be members of multiple patient populations.

Some embodiments generally apply a two-step iterative approach to identify risk factors and triggers for a patient population, and then (based on the identified risk factors and triggers for the patient population) identify risk factors and triggers for an individual patient.

For the first step, the server systems collect and analyze risk factor and disease trigger data from patients in a patient population to identify the risk factors that tend to be most strongly associated with a particular disease symptom for the patients in the patient population. Client devices (under direct or indirect control of the server systems) are configured to prompt patients in the patient population to enter characterization metrics for the risk factors that the server systems have determined to be most strongly associated with the particular disease symptom for the patient population.

For the second step, the server systems analyze the risk factor characterization metrics for the patients in the patient population, and for each patient in the population, the server systems determine the strength of the association (for that patient) between particular risk factors and the disease symptom. Then, for each patient, the server systems designate the risk factors that are most strongly associated with the disease symptom as disease triggers or protectors for individual patients.

This two-step process is iterative in that disease triggers identified for one patient in a patient population can be analyzed for the whole patient population and then tested for individual patients. Some aspects of this iterative, two-phase process are described in PCT Application PCT/US2014/013894 filed on Jan. 30, 2014, the contents of which are incorporated herein by reference. However, other methods for identifying disease triggers/protectors for individual patients could be used instead.

In some embodiments, client devices operated by patients, alone or in combination with external sensors and/or third-party information sources, are configured to monitor and collect data about patient disease symptoms, risk factors, and/or disease triggers and protectors. In operation, the client devices can be configured to: (i) send the collected disease symptom/factor/trigger/protector data directly or indirectly to one or more servers for analysis; and/or (ii) arrange for collected disease symptom/factor/trigger/protector data to be sent to the one or more servers for analysis. The one or more servers in turn, (i) analyze the patient disease symptom/factor/trigger/protector input data received from the client devices, sensors, and/or information sources, and (ii) determine disease triggers and protectors for individual patients, on a patient-by-patient basis.

One difficult challenge with very large sets of data collected from large patient populations (or even a large amount of data collected from even a single patient) is how to organize and display data in ways that allow meaningful conclusions to be drawn from the data. Regardless of the data collection and analysis methods employed, the embodiments disclosed herein enable a researcher (or perhaps a patient) to access the server systems and display at least some of the patient data (e.g., patient disease symptoms, risk factors, and/or disease triggers/protectors) stored therein in one or more intuitive formats. In some embodiments, the intuitive format takes the form of a ladder-style visualization such as the examples shown in FIG. 1 and FIG. 2. However, other visualizations could be used as well.

### BRIEF DESCRIPTION OF THE FIGURES

FIG. 1 shows an example web-based client-server computing system according to some embodiments.

FIG. 2 shows an example client device according to some embodiments.

FIG. 3 shows an example method that includes determining associations and/or correlations between disease factors and a disease symptom for a patient population according to some embodiments.

FIG. 4 shows an example method that includes determining associations and/or correlations between disease factors and/or disease triggers and a disease symptom for a patient according to some embodiments.

FIG. 5 shows an example ladder-style visualization patient data according to some embodiments.

FIG. 6 shows an example ladder-style visualization of patient data according to some embodiments.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, can be arranged, substituted, combined, separated, and designed in a wide variety of different configurations, all of which are explicitly contemplated herein.

### System Overview

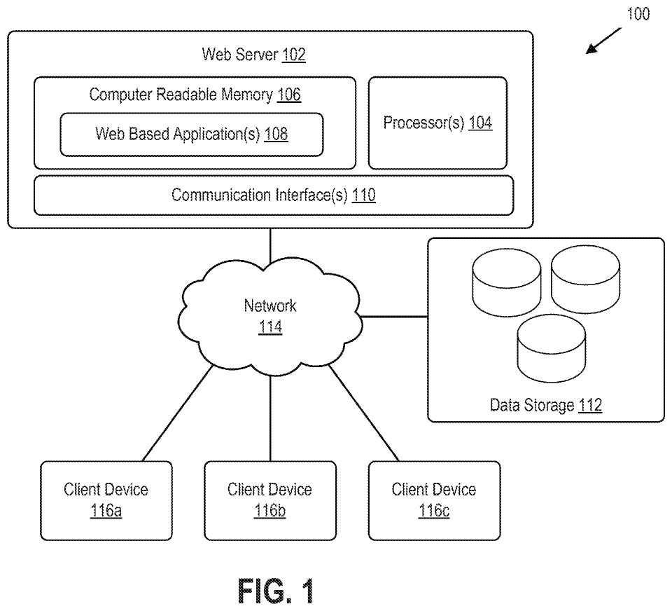

FIG. 1 shows an example web-based client-server computing system 100 according to some embodiments. The example system 100 includes a web server 102 and data storage 112. In operation, the web server 102 is configured to communicate with a plurality of client devices 116a-b over a network 114. In operation, network 114 may comprise one or more of: (i) a local area network (LAN); (ii) a wide area network (WAN); and/or (iii) the Internet or other combination of wired and/or wireless communications networks.

The web server 102 includes one or more processors 104, computer readable memory 106, and one or more communication interfaces 110.

Each of the one or more processor 104 may be any type of processor now known or later developed, including but not limited to a general purpose processor, special purpose processor, application specific integrated circuitry (ASIC), or other type of processor configured to execute computer program instructions.

The computer readable memory 106 may be any type of tangible, non-transitory computer memory now known or later developed, including but not limited to magnetic memory, optical memory, hard discs, optical discs, flash memory, or other type of memory configured to store program code and/or other data. In operation, the computer readable memory 106 is configured to store at least one or more web based applications 108 (or other computing applications), that when executed by the one or more processors 104, cause the web server 102 to perform one or more computing and communications functions, such as the computing and communications functions described herein.

The one or more communication interfaces 110 may be any type of communication interface now known or later developed, including but not limited to wired, wireless, or optical communication interfaces configured to enable the web server 102 to access data storage 112 and to enable the web server to communicate and exchange information with the plurality of client devices 116a-b.

The data storage 112 may be any type of information storage medium, such as computer readable memory. In some embodiments, the data storage 112 is configured as a database system for storing disease symptom, disease factor, and disease trigger data for a plurality of patients and patient populations. In operation, the web server 102 writes data to and reads data from data storage 112 as part of performing the computing and communication functions described herein.

In operation, the web server 102 is configured to receive disease symptom, disease factor, and disease trigger data for individual patients and patient populations, and more particularly, characterization metrics that describe patients' disease symptoms, disease factors, and disease triggers.

Characterization metrics for a patient's disease symptoms/factors/triggers may originate from one or more of a variety of sources, including but not limited to: (i) data that the patient manually enters into his or her client device via a GUI on the client device; (ii) data collected by sensors that are integrated with a patient's client device (e.g., a mobile phone or similar device), including but not limited to integrated optical sensors, cameras, location sensors, motion detectors, gyroscopes, accelerometers, and GPS transceivers; (iii) data collected by medical and/or biometric sensors, that are communicatively coupled to one or both of the patient's client device(s) and/or the web server 102, including but not limited to sensors that detect the patient's temperature, heart rate, blood sugar level, and/or physical activity, e.g., pedometers, thermometers, heart-rate monitors, glucose monitors, or similar sensors/monitors; (iv) data collected by environmental sensors that are communicatively coupled to one or both of the patient's client device(s) and/or the web server 102, including but not limited to thermometers (to measure atmospheric temperature), barometers (to measure air pressure), microphones (to measure ambient sound), optical sensors (to measure light intensity and/or color); and/or (v) data collected from third-party information sources, such as news or weather information services, that are communicatively coupled to one or both of the patient's client device(s) and/or the web server 102, including but not limited to weather, pollen, and/or pollutant data, etc. from servers that provide environmental data related to an area where the patient is located or was located in the past.

The client device(s), biometric sensors, environmental sensors, and third party information sources (collectively, the disease symptom/factor/trigger data sources) may be configured or otherwise instructed to send collected disease symptom/factor/trigger data to the web server 102 in “real-time” (e.g., essentially as soon as the data is available to be sent to the web server 102). Alternatively, the disease symptom/factor/trigger data sources may collect the symptom/factor/trigger data over time, and then periodically send the symptom/factor/trigger data to the web server 102 in batches at regular or semi-regular intervals (every 15 minutes, half hour, hourly, etc.). In some embodiments, certain symptom/factor/trigger data may be identified as “high priority” symptom/factor/trigger data, and the disease symptom/factor/trigger data sources may be configured to send such “high priority” symptom/factor/trigger data to the web server 102 in an expedited fashion. For example, a client device may send “high priority” symptom/factor/trigger data to the web server 102 immediately (or substantially immediately) in response to receiving such symptom/factor/trigger data (or a very short time thereafter) rather than holding and sending such symptom/factor/trigger data to the web server 102 at a later time.

After receiving the symptom/factor/trigger data from any of the above-described disease symptom/factor/trigger data sources, the web server 102 analyzes the received symptom/factor/trigger data to determine one or more of: (i) associations and/or correlations between (i-a) disease symptoms and (i-b) disease factors and/or triggers; (ii) which disease factors are most strongly or highly associated with a particular disease system; and/or (iii) which disease factors are disease triggers for individual patients. Some embodiments generally apply a two-step iterative approach for analyzing the disease symptom/factor/trigger data.

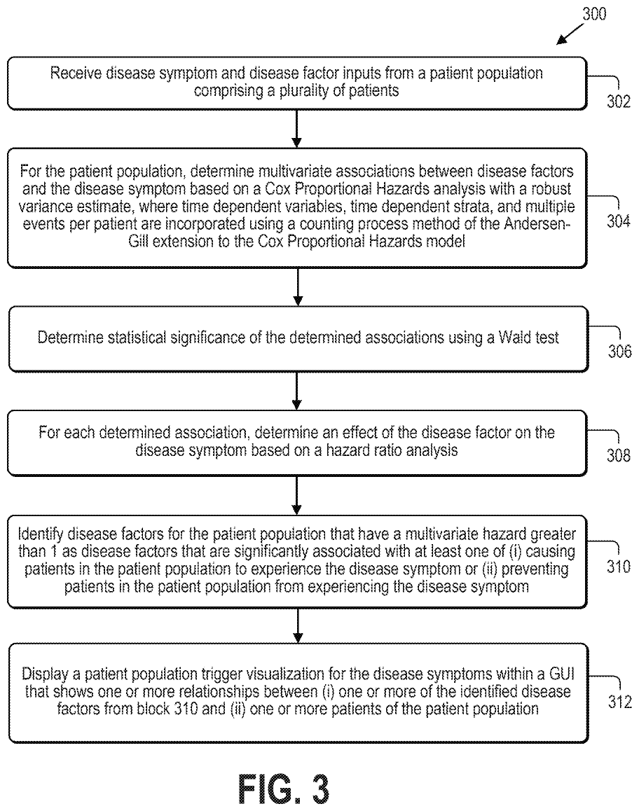

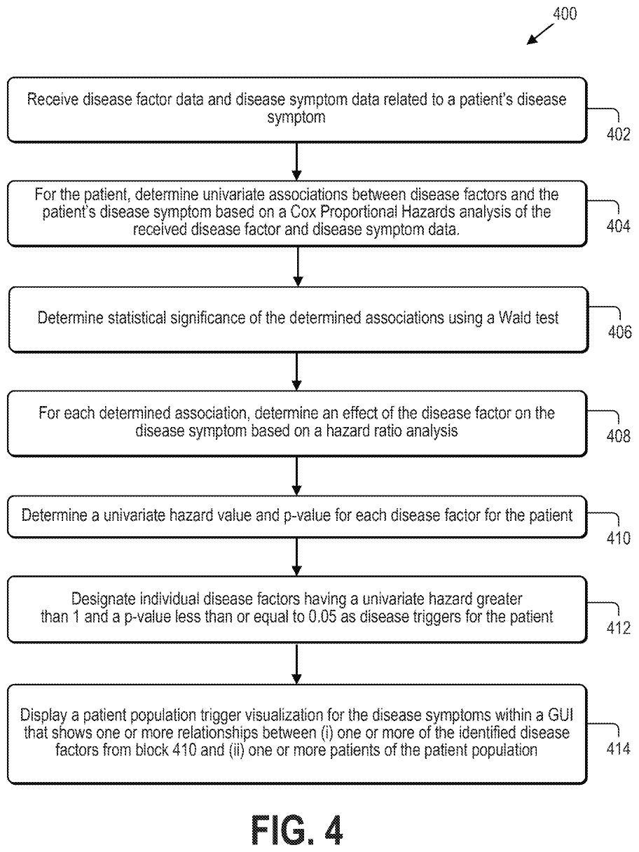

First, the web server 102 analyzes the received symptom/factor/trigger data from all of the patients in a patient population to identify the disease factors and/or triggers that tend to be most strongly associated with a particular disease symptom for the patients in the patient population. Next, the web server 102 analyzes an individual patient's disease symptom/factor/trigger data to one or more of: (i) identify, for that particular patient, which disease factors are most strongly associated with that patient's disease symptom(s); and/or (ii) identify, for that particular patient, which disease factors have a sufficiently strong association with the patient's disease symptom(s) to be identified as a disease trigger for that patient, including, for example, identifying the patient's disease factors/triggers that are most likely to cause the patient to experience a particular disease symptom and/or prevent the patient from experiencing a particular disease symptom. This process is described in more detail with reference to FIGS. 3 and 4.

Because the potential universe of disease factors and triggers is so large, web server 102 can use the disease factors/triggers that are determined to be most strongly associated with a disease symptom for a patient population and/or a particular patient to help determine which actual and/or potential disease factors and disease triggers to focus on.

Each of the client computing devices 116a-b, sometimes referred to herein as client devices or simply clients, may be any of a smartphone, a tablet computer, a laptop computer, a desktop computer, or any other computing device now known or later developed. In operation, individual client devices 116a-c are configured to perform various functions, including but not limited to: (i) receiving, collecting, or otherwise obtaining disease symptom/factor/trigger data from patient inputs and/or sensor readings; (ii) sending disease symptom/factor/trigger data to the web server 102 and/or the data storage 112 (and/or perhaps arranging for disease symptom/factor/trigger data to be sent to the web server 102 and/or the data storage 112); (iii) receiving instructions for prompting patients to enter specific disease symptom/factor/trigger data, and in response, prompting patients to enter the specific disease symptom/factor/trigger data via GUI prompts; (iv) receiving information describing the likelihood that the patient will experience (or not experience) a particular disease symptom in the near future for use with displaying a “risk meter” to the patient, and displaying a “risk meter” within a GUI on the client device(s); and/or (v) receiving information on symptom/factor/trigger associations and disease trigger determinations for use with displaying a “trigger visualization” to the patient or medical professional (e.g., a doctor, researcher, clinician, or other medical professional), and displaying the trigger visualization within a GUI on the client device(s).

Each client device 116a-c typically includes a user-interface, a processor, and/or computer-readable media storing program instructions executable by the processor for performing certain features or functionality described herein. The user-interface may include input devices such as one or more buttons, cameras, microphones, or touchscreens, as well as output devices such as a touchscreen, a display screen, and/or one or more speakers.

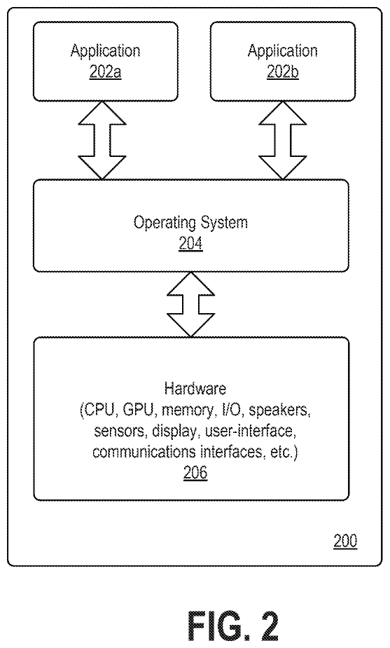

FIG. 2 shows an example client device 200 according to some embodiments. The client device 200 may be similar to or the same as client devices 116a-c shown and described in FIG. 1. In the example of FIG. 2, the client device 200 includes hardware 206 comprising: (i) one or more processors (e.g., a central processing unit(s) or CPU(s) and/or graphics processing unit(s) or GPU(s)); (ii) tangible non-transitory computer readable memory; (iii) input/output components (e.g., speaker(s), sensor(s), display(s), or other interfaces); and (iv) communications interfaces (wireless and/or wired). The hardware 206 components of the client device 102 are configured to run software, including an operating system 204 (or similar) and one or more applications 202a, 202b (or similar) as is known in the computing arts. One or more of the applications 202a and 202b may correspond to program code that, when executed by the one or more processors, cause the client device 200 to perform one or more of the functions and features described herein.

### Determining Associations Between Disease Factors and Disease Symptoms

FIG. 3 shows an example method 300 that includes determining associations and/or correlations between disease factors and a disease symptom for a patient population according to some embodiments, and FIG. 4 shows an example method 400 that includes determining associations and/or correlations between (i) disease factors and/or disease triggers and (ii) a disease symptom for a patient according to some embodiments.

Although the blocks are illustrated in a sequential order, these blocks may in some instances be performed in parallel, and/or in a different order than those described herein. Also, the various blocks may be combined into fewer blocks, divided into additional blocks, and/or removed based on the desired implementation. Additionally, the example methods 300 and 400 describe a server performing the method steps, but in other embodiments, a patient's client device may perform one or more of the method steps.

Also, in methods 300 and 400, each block may represent a module, a segment, or a portion of program code, which includes one or more instructions executable by a processor or computing device for implementing specific logical functions or steps in the method. The program code may be stored on any type of computer readable medium or memory, for example, such as a storage device including a disk or hard drive or other type of memory, such as flash memory or the like. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), and/or flash memory for example. The computer readable media may also be any other volatile or non-volatile storage systems. The computer readable medium may be considered a computer readable storage medium, for example, or a tangible storage device.

In some embodiments, example method 300 is performed by a server system. In such embodiments, the server that performs method 300 may be similar to or the same as any of the servers disclosed and described herein.

Method 300 begins at block 302, which includes receiving disease symptom and disease factor inputs from a patient population comprising a plurality of patients.

After receiving disease symptom and disease factor inputs from the patient population, method 300 advances to block 304, which includes determining (for the patient population) multivariate associations between disease factors and the disease symptom based on a Cox Proportional Hazards analysis with a robust variance estimate, where time dependent variables, time dependent strata, and multiple events per patient are incorporated using a counting process method of the Andersen-Gill extension to the Cox Proportional Hazards model. Some embodiments may alternatively use a logistic regression odds ratio analysis or other statistical methods and/or approaches.

Next, method 300 proceeds to block 306, which includes determining a statistical significance for each of the determined associations using a Wald test. Some embodiments may use alternative methods to determine a statistical significance for each of the determined associations.

After determining the statistical significance of each determined association in block 306, method 300 proceeds to block 308, which includes (for each determined association), determining an effect of the disease factor on the disease symptom based on a hazard ratio or similar analysis.

Next, block 310 includes identifying disease factors for the patient population that have a multivariate hazard greater than 1, and designating those identified disease factors as disease factors that are significantly associated with at least one of (i) causing patients in the patient population to experience the disease symptom, or at least increasing the risk or likelihood that the patients in the patient population will experience the disease symptom, or (ii) preventing patients in the patient population from experiencing the disease symptom, or at least reducing the risk or likelihood that the patients in the patient population will experience the disease symptom.

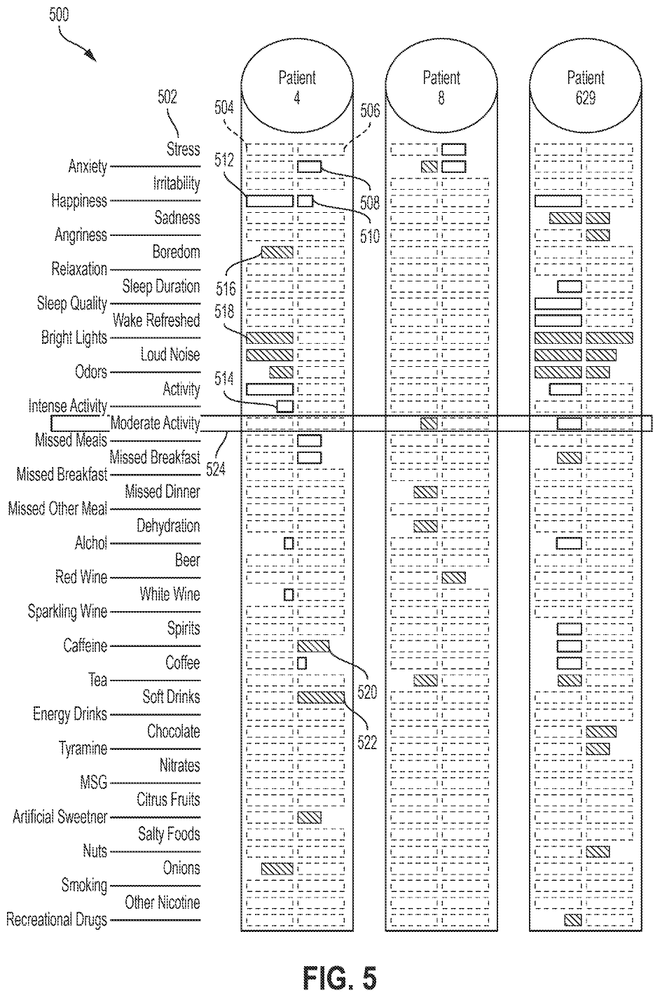

Some embodiments of method 300 may additionally include block 312, which includes displaying a visualization for the disease symptom within a GUI. In operation, the patient population trigger visualization shows one or more relationships between (i) one or more of the identified disease factors from block 310 and (ii) one or more patients of the patient population. In some embodiments, the server system is configured to send data for displaying the patient population trigger visualization to a client device, such as any of the client devices shown and described herein. The patient population trigger visualization may be the same as or similar to the example patient population trigger visualizations shown and described herein with reference to FIGS. 5 and/or 6.

FIG. 4 shows an example method 400 that includes determining associations and/or correlations between (i) disease factors and/or disease triggers and (ii) a disease symptom for a patient according to some embodiments. In some embodiments, method 400 is performed by a server system. In such embodiments, the server that performs method 400 may be similar to or the same as any of the servers disclosed and described herein.

Method 400 begins at block 402, which includes receiving disease factor data and disease symptom data for the individual patient. As described herein, the server system may receive disease factor data and disease symptom data from (i) the patient reporting his or her experience of the disease symptom data and disease factor data via inputs on a client device, (ii) the patient's client device detecting the patient's experience of the disease symptom or disease factors(s) via sensors on or in communication with the client device (e.g., bright lights detected by optical sensors, loud noises detected by microphones, physiological symptoms detected by physiological sensors in communication with the client device), and/or (iii) the server system receiving information about disease factor(s) in the area where the patient is located, e.g., via third party information sources.

Block 404 includes determining univariate associations between disease factors and the patient's disease symptom based on a Cox Proportional Hazards analysis of the received disease factor and disease symptom data. Some embodiments may alternatively use a logistic regression odds ratio analysis or other statistical methods and/or approaches.

At block 406, the server determines, for each determined association, a statistical significance of the determined associations using a Wald test. Some embodiments may use alternatively methods to determine a statistical significance for each of the determined associations.

Next, block 408 includes, for each determined association, determining an effect of the disease factor on the disease symptom based on a hazard ratio analysis or other similar analysis.

Then, at block 410, the server determines a univariate hazard value and p-value for each disease factor for the patient.

Next, at block 412, the server designates individual disease factors having a univariate hazard greater than 1 and a p-value less than or equal to 0.05 (or perhaps some other p-value threshold) as disease triggers for that particular patient. In some embodiments, the patient's identified disease triggers may be displayed within a trigger visualization for the patient, such as the trigger visualizations shown and described herein with reference to FIGS. 5 and/or 6.

Some embodiments may additionally include block 414, where the server displays a patient population trigger visualization for the disease symptom within a GUI. In operation, the patient population trigger visualization shows relationships between (i) one or more of the disease triggers determined in block 412 and (ii) one or more patients of the patient population.

In some embodiments, the server system is configured to send data for displaying the patient population trigger visualization to a client device, such as any of the client devices shown and described herein. The patient population trigger visualization may be the same as or similar to the example patient population trigger visualizations shown and described herein with reference to FIGS. 5 and/or 6.

### Example Visualizations

FIG. 5 shows an example ladder-style visualization 500 of at least a portion of some patient data (e.g., patient disease symptoms, risk factors, and/or disease triggers/protectors) indicating whether and the extent to which individual risk factors from a set of risk factors 302 for a first disease symptom (e.g., migraine headache) and a second disease symptom (e.g., non-migraine headache) are disease triggers or protectors for three patients: patients 4, 8, and 629. Although visualization 500 shows a comparison for three patients, visualization 500 could show data for one, two, three, or many more patients.

Whether and the extent to which a particular risk factor is a disease trigger or protector for the first disease symptom is shown by blocks in column 504, and whether and the extent to which a particular risk factor is a disease trigger or protector for the second disease symptom is shown by blocks in column 506. Patients 4, 8, and 629 could all be in the same patient population (described above) but they need not necessarily be in the same patient population.

The left side of visualization 500 lists the set of risk factors 502, including stress, anxiety, irritability, etc. In some embodiments, the set of risk factors 502 may include more, fewer, and/or different risk factors than the set of risk factors 502 shown in example visualization 500. For example, in some embodiments, the set of risk factors 502 may include about 70 different risk factors. Similarly, different disease symptoms tend to have different risk factors.

Column 504 includes a set of boxes, where each individual box in column 504 corresponds to a specific risk factor in the set of risk factors 502. The size and color of the block (or lack of a block) in each box in column 504 shows, for patient 4, (i) whether the corresponding risk factor is a disease trigger or protector (or neither) for the first disease symptom, and (ii) the degree or “strength” of the association between that box's corresponding risk factor and the first disease symptom for the patient (e.g., the Cox Hazard Ratio, logistic regression odds ratio, p-value, or other quantification of the association).

Similarly, column 506 also includes a set of boxes, where each individual box in column 506 corresponds to a specific risk factor in the set of risk factors 502. The size and color of the block (or lack of a block) in each box in column 506 shows, for patient 4, (i) whether the corresponding risk factor is a disease trigger or protector (or neither) for the second disease symptom, and (ii) the degree or “strength” of the association between that box's corresponding risk factor and the second disease symptom for the patient (e.g., the Cox Hazard Ratio, logistic regression odds ratio, p-value, or other quantification of the association).

In the example visualization 500, the first disease symptom (column 504) is migraine headache and the second disease symptom (column 506) is non-migraine headache. Although two columns are shown for patient 4 (and each of the other patients), other embodiments could include additional columns for additional disease symptoms for an individual patient. Also, a purple block indicates that a particular risk factor is a disease trigger, a blue block indicates that a particular risk factor is a protector, and the lack of a colored block indicates that a particular risk factor is neither a disease trigger nor protector. Additionally, the size (length in this example) represents the strength of the statistical association (ranging from p≤0.5 to p≥0.001). However, other colors, indicators, and correlations (e.g., other than size) could be used as well.

In visualization 500, for migraine headaches indicated by column 504, blue block 512 indicates that happiness is a protector against migraine headaches for patient 4. Similarly blue block 514 indicates that intense activity is also a protector against migraine headache for patient 4. Blue block 512 is larger/longer than blue block 514, which shows that, for patient 4, happiness is a stronger protector against migraine headache than intense activity.

Additionally, purple block 516 indicates that boredom is a disease trigger for migraine headache for patient 4. Similarly, purple block 518 indicates that bright lights are also a disease trigger for migraine headache for patient 4. Purple block 518 is larger/longer than purple block 516, which shows that, for patient 4, bright lights are a stronger disease trigger for migraine headache than boredom.

Further, the lack of a blue or purple block for stress, irritability, sparkling wine, chocolate, and many other risk factors in column 504 indicates either (i) stress, irritability, sparkling wine, and chocolate (as well as any other risk factor without a corresponding blue or purple block) are not disease triggers or protectors for migraine headache for patient 4, or (ii) there is insufficient data for the server system to conclude that stress, irritability, sparkling wine, and chocolate (as well as any other risk factor without a corresponding blue or purple block) are disease triggers or protectors for migraine headache for patient 4. Some embodiments may use different colors to distinguish between (i) risk factors which have been statistically established as not being either a disease trigger or protector versus (ii) risk factors that lack sufficient data to conclude whether they are disease triggers or protectors.

The colored blocks (or lack thereof) in column 506 for non-migraine headache are similar to the colored blocks (or lack thereof) in column 504 for migraine headache.

For example, for non-migraine headaches indicated by column 506, blue block 508 indicates that anxiety is a protector against non-migraine headache for patient 4. Similarly, blue block 510 indicates that happiness is also a protector against non-migraine headache for patient 4. Blue block 508 is slightly larger/longer than blue block 510, which shows that, for patient 4, anxiety is a stronger protector against non-migraine headache than happiness.

Additionally, purple block 520 indicates that caffeine is a disease trigger for non-migraine headache for patient 4. And similarly, purple block 522 indicates that soft drinks are also a disease trigger for non-migraine headache for patient 4. Purple block 522 is larger/longer than purple block 520, which shows that, for patient 4, soft drinks are a stronger disease trigger for non-migraine headaches than caffeine.

Further, the lack of a blue or purple block for stress, irritability, sparkling wine, chocolate, and many other risk factors in column 506 indicates that that either (i) stress, irritability, sparkling wine, and chocolate (as well as any other risk factor without a corresponding blue or purple block) are not disease triggers or protectors for non-migraine headache for patient 4, or (ii) there is insufficient data for the server system to conclude that stress, irritability, sparkling wine, and chocolate (as well as any other risk factor without a corresponding blue or purple block) are disease triggers or protectors for non-migraine headache for patient 4. Some embodiments may use different colors to distinguish between (i) risk factors which have been statistically established as not being either a disease trigger or protector versus (ii) risk factors that lack sufficient data to conclude whether they are disease triggers or protectors.

By displaying the disease trigger and protector data for risk factors for two different disease symptoms (e.g., migraine headache in column 504 and non-migraine headache in column 506) side-by-side for patient 4, visualization 500 shows a researcher (or perhaps patient 4 or even other patients) the relationships, or perhaps lack thereof, between risk factors for migraine and non-migraine headaches for an individual patient.

Additionally, by displaying disease trigger and protector data for risk factors for two different disease symptoms (e.g., migraine headache and non-migraine headache) side-by-side for multiple patients (i.e., patients 4, 8, and 629), visualization 500 shows a researcher (or perhaps one or more patients) the relationships, or perhaps lack thereof, between risk factors for migraine and non-migraine headaches for multiple patients.

For example, box 524 shows how risk factor “moderate activity” differs for migraine and non-migraine headaches for patients 4, 8, and 629. In particular, moderate activity is (i) neither a disease trigger for nor a protector against either migraine or non-migraine headaches for patient 4, (ii) a disease trigger for migraine headache for patient 8, but neither a disease trigger for nor protector against non-migraine headache for patient 8, and (iii) a protector against migraine headache for patient 629, but neither a disease trigger for nor protector against non-migraine headache for patient 629.

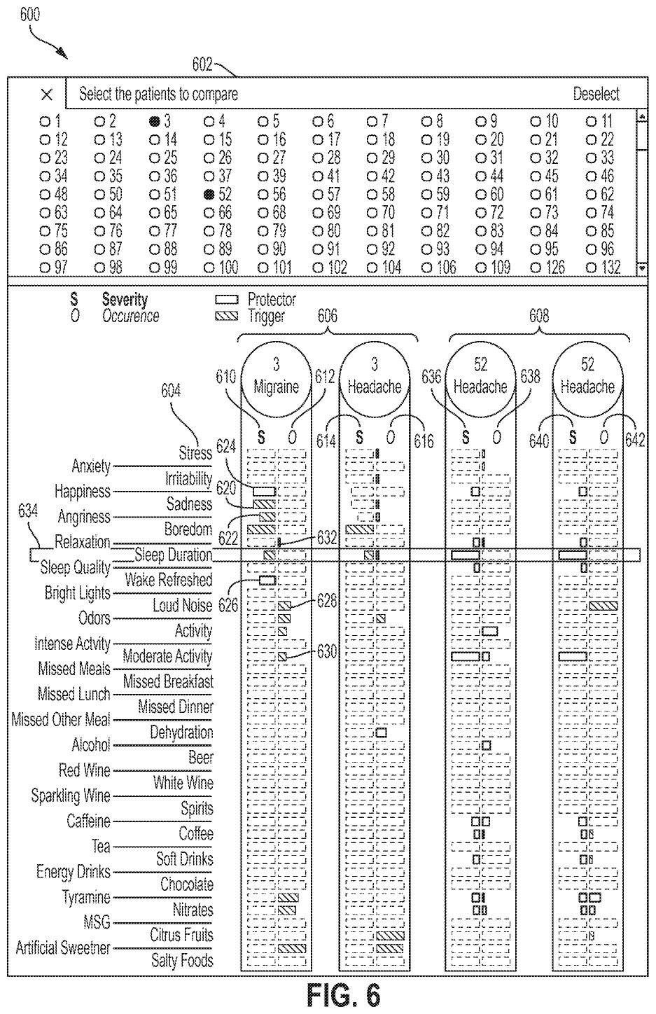

FIG. 6 shows another example ladder-style visualization 600 of at least a portion of some patient data (e.g., patient disease symptoms, risk factors, and/or disease triggers/protectors) indicating whether and the extent to which individual risk factors from a set of risk factors 604 for a first disease symptom (e.g., migraine headache) and a second disease symptom (e.g., non-migraine headache) are disease triggers or protectors for two patients: patients 3 and 52. Data set 606 is patient data for patient 3, and data set 608 is patient data for patient 52. Although visualization 600 shows a comparison between two patients, visualization 600 could show data for one, two, three, or many more patients.

Visualization 600 is similar to visualization 500 except that visualization 600 additionally shows whether and the extent to which a particular risk factor affects the onset or severity (or perhaps both) of one or more disease symptoms. Visualization 600 shows two disease symptoms as an example: (i) migraine headache and (ii) non-migraine headache. However, visualization 600 could be used with more, fewer, and/or different disease symptoms than the ones shown in FIG. 6.

In visualization 600, blocks in column 610 show whether and the extent to which a particular risk factor is a disease trigger for or protector against the severity of migraine headache, and blocks in column 612 show whether and the extent to which a particular risk factor is a disease trigger for or protector against the onset of migraine headache. Similarly, blocks in column 614 show whether and the extent to which a particular risk factor is a disease trigger for or protector against the severity of non-migraine headache, and blocks in column 616 show whether and the extent to which a particular risk factor is a disease trigger for or protector against the onset of non-migraine headache.

Visualization 600 includes similar columns for the severity and onset of migraine and non-migraine headache for patient 52, too. In particular blocks in column 636 show whether and the extent to which a particular risk factor is a disease trigger for or protector against the severity of migraine headache, and blocks in column 638 show whether and the extent to which a particular risk factor is a disease trigger for or protector against the onset of migraine headache. Similarly, blocks in column 640 show whether and the extent to which a particular risk factor is a disease trigger for or protector against the severity of non-migraine headache, and blocks in column 642 show whether and the extent to which a particular risk factor is a disease trigger for or protector against the onset of non-migraine headache

Patients 3 and 52 could both be in the same patient population (described above) but they need not necessarily be in the same patient population. Similarly, visualization 600 could include many more than two patients. For example, selection block 602 at the top of visualization 600 allows a user (such as a researcher or patient) to select individual patients for comparison. As shown in selection block 602, patients 3 and 52 have been selected, which is why patient data for patients 3 and 52 are shown in the main window of visualization 600.

The left side of visualization 600 lists the set of risk factors 604, including stress, anxiety, irritability, etc. In some embodiments, the set of risk factors 604 may include more, fewer, and/or different risk factors than the risk factors shown in the set of risk factors 604. For example, in some embodiments, the set of risk factors 604 may include about 70 different risk factors. The risk factors in column 604 may be the same or substantially the same as the set of risk factors 502 shown and described with reference to FIG. 5.

Column 610 includes a set of boxes, where each individual box in column 610 corresponds to a specific risk factor in the set of risk factors 604. The color of the block (or lack of a block) in each box in column 610 shows whether the corresponding risk factor is a disease trigger or protector (or neither) for the severity of migraine headache for patient 3, i.e., whether the risk factor tends to increase or reduce the severity of migraine for patient 3. The absence of a colored block in column 610 for a particular risk factor shows that the risk factor is neither a disease trigger nor a protector for patient 3, with respect to migraine severity, or at least that the server system does not have sufficient data to conclude that the risk factor is a disease trigger or protector for patient 3, with respect to migraine severity. And for those risk factors with a colored block in column 610, the size of the colored block indicates the extent to which the risk factor tends to increase or reduce the severity of migraine for patient 3.

Column 612 includes a set of boxes, where each individual box in column 612 corresponds to a specific risk factor in the set of risk factors 604. The color of the block (or lack of a block) in each box in column 612 shows whether the corresponding risk factor is a disease trigger or protector (or neither) for the onset of migraine headache for patient 3, i.e., whether the risk factor tends to increase or reduce the likelihood of onset of migraine for patient 3. The absence of a colored block in column 612 for a particular risk factor shows that the risk factor is neither a disease trigger nor a protector for patient 3, with respect to migraine onset, or at least that the server system does not have sufficient data to conclude that the risk factor is a disease trigger or protector for patient 3, with respect to migraine onset. And for those risk factors with a colored block in column 612, the size of the colored block indicates the extent to which the risk factor tends to increase or reduce the likelihood of onset of migraine for patient 3.

The colored blocks (or lack thereof) in columns 614 and 616 for non-migraine headache severity and onset, respectively, are similar to the colored blocks (or lack thereof) in columns 610 and 612 for migraine headache severity and onset, respectively.

In particular, column 614 includes a set of boxes, where each individual box in column 614 corresponds to a specific risk factor in the set of risk factors 604. The color of the block (or lack of a block) in each box in column 614 shows whether the corresponding risk factor is a disease trigger or protector (or neither) for the severity of non-migraine headache for patient 3, i.e., whether the risk factor tends to increase or reduce the severity of a non-migraine for patient 3. The absence of a colored block in column 614 for a particular risk factor shows that the risk factor is neither a disease trigger nor a protector for patient 3, with respect to non-migraine headache severity, or at least that the server system does not have sufficient data to conclude that the risk factor is a disease trigger or protector for patient 3, with respect to non-migraine headache severity. And for those risk factors with a colored block in column 614, the size of the colored block indicates the extent to which the risk factor tends to increase or reduce the severity of a non-migraine headache for patient 3.

Column 616 includes a set of boxes, where each individual box in column 616 corresponds to a specific risk factor in the set of risk factors 604. The color of the block (or lack of a block) in each box in column 616 shows whether the corresponding risk factor is a disease trigger or protector (or neither) for the onset of non-migraine headache, i.e., whether the risk factor tends to increase or reduce the likelihood of onset of non-migraine headache for patient 3. The absence of a colored block in column 616 for a particular risk factor shows that the risk factor is neither a disease trigger nor a protector for patient 3, with respect to non-migraine headache onset, or at least that the server system does not have sufficient data to conclude that the risk factor is a disease trigger or protector for patient 3, with respect to non-migraine headache onset. And for those risk factors with a colored block in column 616, the size of the colored block indicates the extent to which the risk factor tends to increase or reduce the likelihood of onset of non-migraine headache for patient 3.

Although visualization 600 shows the first disease symptom as migraine headache and the second disease symptom as non-migraine headache, additional or alternative disease symptoms could be displayed as well. Also, although visualization 600 uses a purple block to indicate that a particular risk factor is a disease trigger, a blue block to indicate that a particular risk factor is a protector, and the lack of a colored block to indicate that a particular risk factor is neither a disease trigger nor protector, other colors or indications could be used instead. Additionally, the size (length in this example) represents the strength of the statistical association (ranging from p≤0.5 to p≥0.001). However, other colors, indicators, and correlations (e.g., other than size) could be used as well.

In visualization 600, for migraine headache severity indicated by column 610, purple block 620 indicates that sadness is a trigger for migraine headaches for patient 3, i.e., that sadness tends to increase the severity of a migraine headache for patient 3. Similarly purple block 620 indicates that angriness is also a trigger for migraine headache for patient 3, i.e., that angriness tends to increase the severity of a migraine headache for patient 3. Purple block 620 is larger than purple block 622, which shows that, for patient 3, sadness affects the severity of migraine headache more than angriness.

Additionally, blue block 624 indicates that happiness is a protector against migraine headache severity for patient 3, i.e., that happiness tends to reduce the severity of a migraine for patient 3. Similarly, blue block 626 indicates that wake refreshed is also a protector against migraine headache severity for patient 3, i.e., that wake refreshed tends to reduce migraine severity for patient 3. Blue block 624 is larger than blue block 626, which shows that, for patient 3, happiness affects the severity of migraine headache more than wake refreshed. Here, happiness tends to reduce the severity of a migraine for patient 3 more than wake refreshed.

Further, the lack of a blue or purple block for stress, alcohol, chocolate, and many other risk factors in column 610 indicates that either (i) stress, alcohol, and chocolate (as well as any other risk factor without a corresponding blue or purple block in column 610) are not disease triggers for or protectors against the severity of migraine headache for patient 3, or (ii) there is insufficient data for the server system to conclude whether or the extent to which stress, alcohol, and chocolate (as well as any other risk factor without a corresponding blue or purple block in column 610) affect the severity of migraine headache for patient 3.

Column 612 is similar to column 610 except that column 612 shows whether and the extent to which individual risk factors affect the onset of migraine headache for patient 3 whereas column 610 shows whether and the extent to which individual risk factors affect the severity of migraine headache for patient 3.

For example, for migraine headache onset indicated by column 612, purple block 628 indicates that loud noise is a trigger for migraine headache onset for patient 3, i.e., that loud noise tends to increase the likelihood of migraine headache onset for patient 3. Similarly purple block 630 indicates that moderate activity is also a trigger for migraine headache onset for patient 3, i.e., that moderate activity tends to increase the likelihood of migraine headache onset for patient 3. Purple block 628 is larger than purple block 630, which shows that, for patient 3, loud noise increases the likelihood of migraine headache onset more than moderate activity.

Additionally, blue block 632 indicates that relaxation is a protector against migraine headache onset for patient 3, i.e., that relaxation tends to reduce the likelihood migraine headache onset for patient 3.

Further, the lack of a blue or purple block for stress, alcohol, chocolate, and many other risk factors in column 612 indicates that either (i) stress, alcohol, and chocolate (as well as any other risk factor without a corresponding blue or purple block in column 612) are not disease triggers for or protectors against the onset of migraine headache for patient 3, or (ii) there is insufficient data for the server system to conclude whether or the extent to which stress, alcohol, and chocolate (as well as any other risk factor without a corresponding blue or purple block in column 612) tends to increase or decrease the likelihood of migraine headache onset for patient 3.

By displaying the disease trigger and protector data for risk factors for both the severity and onset of two different disease symptoms (e.g., migraine headache severity in column 610, migraine headache onset in column 612, non-migraine headache severity in column 614, and non-migraine headache onset in column 616) side-by-side for patient 3, visualization 600 shows a researcher (or perhaps patient 3 or even other patients) the relationships, or perhaps lack thereof, between risk factors for the severity and onset of migraine and non-migraine headaches for an individual patient.

Additionally, by displaying whether and the extent to which specific risk factors affect the severity and onset of multiple disease symptoms side-by-side for multiple patients, researchers and/or patients can readily assess the relationships, or perhaps lack thereof, between risk factors for migraine severity and onset and non-migraine headache severity and onset for multiple patients, or even a patient population.

Visualizations 500 and 600 enable researchers (and/or patients) to review and consider (i) how a particular patient's disease triggers and protectors compare with other patients within and/or outside of that particular patient's patient population, (ii) whether and the extent to which certain disease triggers or protectors may be more or less prevalent within a particular patient population, both in terms of onset and severity of a disease symptom, and/or (iii) whether and the extent to which a patient may have more or fewer disease triggers as compared to other patients within or outside of that patient's patient population. As mentioned previously, a patient population may include many (hundreds, thousands, or perhaps millions) of patients who all share one or more similarities (e.g., the same age or age range, same gender, same ethnicity, same national origin, suffer from the same disease, have the same allergies, have the same genetic markers, and/or perhaps other similarities). Some patients may be members of multiple patient populations.

For example, block 634 shows how sleep duration affects patients 3 and 52 for migraine severity, migraine onset, non-migraine headache severity, and non-migraine onset.

In particular, the purple block in column 610 for sleep duration shows that sleep duration is a trigger for migraine severity for patient 3, i.e., that sleep duration tends to increase severity of a migraine for patient 3. The lack of a block in column 612 for sleep duration shows that sleep duration does not affect migraine onset for patient 3, or at least that there is insufficient data to conclude whether or the extent to which sleep duration affects migraine onset for patient 3. The purple block in column 614 for sleep duration shows that sleep duration is a trigger for non-migraine headache severity for patient 3, i.e., that sleep duration tends to increase severity of a non-migraine headache for patient 3. The small blue block in column 616 for sleep duration shows that sleep duration is a protector against the onset of non-migraine headaches for patient 3, i.e., that sleep duration tends to reduce the likelihood of non-migraine headache onset for patient 3.

Similarly, the blue block in column 636 for sleep duration shows that sleep duration is a protector against migraine severity for patient 52, i.e., that sleep duration tends to reduce the severity of a migraine for patient 52. The lack of a block in column 638 for sleep duration shows that sleep duration does not affect migraine onset for patient 52, or at least that there is insufficient data to conclude whether or the extent to which sleep duration affects migraine onset for patient 52. The blue block in column 640 for sleep duration shows that sleep duration is a protector against non-migraine headache severity for patient 52, i.e., that sleep duration tends to reduce the severity of a non-migraine headache for patient 52. And the lack of a block in column 642 for sleep duration shows that sleep duration does not affect non-migraine headache onset for patient 52, or at least that there is insufficient data to conclude whether or the extent to which sleep duration affects the onset of non-migraine headaches for patient 52.

In some embodiments, visualizations 500 and/or 600 may additionally include or otherwise be associated with one or more input fields (not shown) that enable trigger and protector data to be sorted, filtered, and/or analyzed on one or more of a number of factors, including but not limited to patient, patient population, gender, age, age range, geographic location, ethnicity, national origin, type or location of employment, route of travel, medical treatment, genetic marker, disease symptom, disease symptom severity, disease symptom frequency, disease trigger, and disease protector. In operation, the sorted and/or filtered data can help identify similarities in disease symptom manifestation and disease symptoms/protectors for individual patients and/or patient populations, or perhaps facilitate groupings of patients or patient populations into different sets for display and analysis.

The foregoing summary is illustrative only and is not intended to be in any way limiting. In addition to the illustrative aspects, embodiments, and features described above, further aspects, embodiments, and features will become apparent by reference to the figures and corresponding technical description.

What is claimed is:

1. A method comprising:
receiving disease symptom and disease factor inputs from a patient population comprising a plurality of patients;
for the patient population, determining multivariate associations between disease factors and the disease symptom based on a Cox Proportional Hazards analysis with a robust variance estimate, where time dependent variables, time dependent strata, and multiple events per patient are incorporated using a counting process method of the Andersen-Gill extension to the Cox Proportional Hazards model;
determining one or more statistical significances of the determined associations using a Wald test;
for each determined association, determine an effect of the disease factor on the disease symptom based on a hazard ratio analysis;
identifying disease factors for the patient population that have a multivariate hazard greater than 1 as disease factors that are significantly associated with at least one of (i) causing patients in the patient population to experience the disease symptom or (ii) preventing patients in the patient population from experiencing the disease symptom;
causing a graphical user interface to display a patient population trigger visualization for the disease symptoms, wherein the trigger visualization comprises a plurality of rows and one or more columns, wherein a first column corresponds to a first disease symptom for a first patient, and wherein a first row in the first column comprises an indication of an extent to which a first risk factor is a disease trigger or disease protector for the first disease symptom for the first patient.

2. The method of claim 1, wherein the trigger visualization further comprises a second column for the first patient, wherein the second column corresponds to a second disease symptom for a first patient, and wherein the first row in the second column comprises an indication of an extent to which the first risk factor is a disease trigger or disease protector for the second disease symptom for the first patient.

3. The method of claim 1, wherein the trigger visualization further comprises a third column for a second patient, wherein the third column corresponds to the first disease symptom for the second patient, and wherein the first row in the third column comprises an indication of an extent to which the first risk factor is a disease trigger or disease protector for the first disease symptom for the second patient.

4. The method of claim 1, wherein the trigger visualization further comprises a fourth column for the second patient, wherein the fourth column corresponds to a second disease symptom for the second patient, and wherein the first row in the fourth column comprises an indication of an extent to which the first risk factor is a disease trigger or disease protector for the second disease symptom for the second patient.

5. The method of claim 1, wherein the first row in the first column comprises an indication of an extent to which the first risk factor positively or negatively affects a severity of the first disease symptom for the first patient, wherein the trigger visualization further comprises a second column for the first patient, and wherein the first row in the second column comprises an indication of an extent to which the first risk factor positively or negatively affects an occurrence of the first disease symptom for the first patient.

6. The method of claim 5, wherein the trigger visualization further comprises a third column for the first patient and a fourth column for the first patient, wherein the third column and the fourth column correspond to a second disease symptom for the first patient, wherein the first row in the third column comprises an indication of an extent to which the first risk factor positively or negatively affects a severity of the second disease symptom for the first patient, and wherein the first row in the fourth column comprises an indication of an extent to which the first risk factor positively or negatively affects an occurrence of the second disease symptom for the first patient.

7. The method of claim 5, wherein the trigger visualization further comprises a fifth column for a second patient and a sixth column for the second patient, wherein the fifth column and the sixth column correspond to the first disease symptom for the second patient, wherein the first row in the fifth column comprises an indication of an extent to which the first risk factor positively or negatively affects a severity of the first disease symptom for the second patient, and wherein the first row in the sixth column comprises an indication of an extent to which the first risk factor positively or negatively affects an occurrence of the first disease symptom for the second patient.

8. The method of claim 7, wherein the trigger visualization further comprises a seventh column for the second patient and an eighth column for the second patient, wherein the seventh column and the eighth column correspond to the second disease symptom for the second patient, wherein the first row in the sixth column comprises an indication of an extent to which the first risk factor positively or negatively affects a severity of the second disease symptom for the second patient, and wherein the first row in the eighth column comprises an indication of an extent to which the first risk factor positively or negatively affects an occurrence of the second disease symptom for the second patient.

9. Tangible, non-transitory computer-readable media comprising instructions stored therein, wherein the instructions, when executed by one or more processors, cause one or more computing systems to perform a method comprising:
receiving disease symptom and disease factor inputs from a patient population comprising a plurality of patients;
determining whether individual disease factors tend to (i) cause individual patients in the patient population to experience individual disease symptoms or (ii) prevent individual patients in the patient population from experiencing individual disease symptoms;
causing a graphical user interface to display a patient population trigger visualization for the disease symptoms, wherein the trigger visualization comprises a plurality of rows and one or more columns, wherein a first column corresponds to a first disease symptom for a first patient, and wherein a first row in the first column comprises an indication of an extent to which a first risk factor is a disease trigger or disease protector for the first disease symptom for the first patient.

10. The tangible, non-transitory computer-readable media of claim 9, wherein the trigger visualization further comprises a second column for the first patient, wherein the second column corresponds to a second disease symptom for a first patient, and wherein the first row in the second column comprises an indication of an extent to which the first risk factor is a disease trigger or disease protector for the second disease symptom for the first patient.

11. The tangible, non-transitory computer-readable media of claim 9, wherein the trigger visualization further comprises a third column for a second patient, wherein the third column corresponds to the first disease symptom for the second patient, and wherein the first row in the third column comprises an indication of an extent to which the first risk factor is a disease trigger or disease protector for the first disease symptom for the second patient.

12. The tangible, non-transitory computer-readable media of claim 9, wherein the trigger visualization further comprises a fourth column for the second patient, wherein the fourth column corresponds to a second disease symptom for the second patient, and wherein the first row in the fourth column comprises an indication of an extent to which the first risk factor is a disease trigger or disease protector for the second disease symptom for the second patient.

13. The tangible, non-transitory computer-readable media of claim 9, wherein the first row in the first column comprises an indication of an extent to which the first risk factor positively or negatively affects a severity of the first disease symptom for the first patient, wherein the trigger visualization further comprises a second column for the first patient, and wherein the first row in the second column comprises an indication of an extent to which the first risk factor positively or negatively affects an occurrence of the first disease symptom for the first patient.

14. The tangible, non-transitory computer-readable media of claim 13, wherein the trigger visualization further comprises a third column for the first patient and a fourth column for the first patient, wherein the third column and the fourth column correspond to a second disease symptom for the first patient, wherein the first row in the third column comprises an indication of an extent to which the first risk factor positively or negatively affects a severity of the second disease symptom for the first patient, and wherein the first row in the fourth column comprises an indication of an extent to which the first risk factor positively or negatively affects an occurrence of the second disease symptom for the first patient.

15. The tangible, non-transitory computer-readable media of claim 13, wherein the trigger visualization further comprises a fifth column for a second patient and a sixth column for the second patient, wherein the fifth column and the sixth column correspond to the first disease symptom for the second patient, wherein the first row in the fifth column comprises an indication of an extent to which the first risk factor positively or negatively affects a severity of the first disease symptom for the second patient, and wherein the first row in the sixth column comprises an indication of an extent to which the first risk factor positively or negatively affects an occurrence of the first disease symptom for the second patient.

16. The tangible, non-transitory computer-readable media of claim 15, wherein the trigger visualization further comprises a seventh column for the second patient and an eighth column for the second patient, wherein the seventh column and the eighth column correspond to the second disease symptom for the second patient, wherein the first row in the sixth column comprises an indication of an extent to which the first risk factor positively or negatively affects a severity of the second disease symptom for the second patient, and wherein the first row in the eighth column comprises an indication of an extent to which the first risk factor positively or negatively affects an occurrence of the second disease symptom for the second patient.
